(19) United States
(12) Patent Application Publication
(10) Pub. No.: US 2021/0085398 A1
(43) Pub. Date: Mar. 25, 2021

(54) METHOD AND SYSTEM FOR SUPPORTING MEDICAL PERSONNEL DURING A RESECTION, AND COMPUTER PROGRAM PRODUCT

(71) Applicant: Siemens Healthcare GmbH, Erlangen (DE)

(72) Inventors: Amilcar Alzaga, Nürnberg (DE); Alois Regensburger, Erlangen (DE); Sabrina Walter, Bamberg (DE); Michael Wiets, Langensendelbach (DE)

(21) Appl. No.: 17/017,803

(22) Filed: Sep. 11, 2020

(30) Foreign Application Priority Data
Sep. 19, 2019 (DE) 102019214303.2

(51) Int. Cl. A61B 34/10; A61B 34/20; A61B 90/00

(52) U.S. Cl. CPC A61B 34/10; A61B 34/20; A61B 90/37; A61B 2034/2063; A61B 2034/107; A61B 2034/102; A61B 2090/378

(57) ABSTRACT

Methods, systems, and computer program products are provided for supporting medical personnel during a resection. In the method, a preoperative three-dimensional (3D) data set of an examination object on which the resection is to be carried out is acquired. Furthermore, a resection surface is acquired by ultrasound to generate an ultrasound data set describing the resection surface. The ultrasound data set is registered with the respective current 3D data set. The current 3D data set is updated with the resection surface acquired by ultrasound.

The present patent document claims the benefit of German Patent Application No. 10 2019 214 303.2, filed Sep. 19, 2019, which is hereby incorporated by reference.

### TECHNICAL FIELD

The present disclosure relates to a method for supporting medical personnel during a resection, a corresponding computer program product, and a system for carrying out such a method.

### BACKGROUND

Pre-operative imaging has become established as an important part of device-assisted and computer-assisted medicine. Pre-operatively obtained image data may serve, for example, as the basis for a pre-operative planning and for an intraoperative navigation. As soon as a resection has been started in the context of the respective operation or intervention, albeit then a resection, (e.g., of a liver tumor), the pre-operative image data or a three-dimensional (3D) model generated therefrom, (e.g., of the liver), is no longer current because the actually performed resection is not represented therein.

A conventional approach for estimating an intraoperative incision surface uses optical stereo reconstruction in which a stereoscopic camera image of the incision surface is recorded, and an attempt is made to create therefrom a respective current 3D model. This approach may however result, at least according to the current state of the art, in satisfactory results only in solid, that is, substantially undeformable or at least almost unmovable organs, (e.g., the brain), whereas in soft, deformable, and/or movable or displaceable organs, (e.g., the liver), it is bound to fail. In the case of such organs, based on camera images, it cannot be reliably recognized whether the organ has merely been moved or deformed or whether a recognizable change is attributable to a resection or a further incision or tissue removal. Purely geometrical estimations of how deeply an instrument has already penetrated into the tissue or a use of a deformation model herein also cannot always produce reliable results or evaluations and/or are associated with a significant effort.

A further approach lies in the use of intraoperative imaging by magnetic resonance tomography (MRT) or contrasted computed tomography (CT). Both are very cost and time-intensive and may represent an additional burden for the respective patient. In addition, such imaging methods may not be used without interrupting the respective intervention, that is, they may only be used stepwise.

### SUMMARY AND DESCRIPTION

It is an object of the present disclosure to provide an improved possibility for technical support to medical personnel during a resection. The scope of the present disclosure is defined solely by the appended claims and is not affected to any degree by the statements within this summary. The present embodiments may obviate one or more of the drawbacks or limitations in the related art.

A method serves for supporting medical personnel during a resection. The method may thus be applied in parallel with such an intervention but does not concern surgical acts or measures carried out therein but is restricted to the processing and provision of data and the technical support for the personnel. The method may thus be carried out, in principle, independently of an actual resection, (e.g., based on model data provided or in parallel with a processing of non-living tissue or material).

In a method act of the method, a pre-operative 3D database or a 3D model of an examination object correspondingly created therefrom on which the resection is to be performed, is acquired. The 3D data set may thus represent a three-dimensional image representation of the examination object, for example, in the form of magnetic resonance or computed tomography data, or a corresponding reconstruction or the like. The examination object may be a patient, a part of a patient such as an organ or a portion or subregion of an organ, a tissue sample, or an object or material accessible to the 3D imaging. Particularly usefully, the method may be used for soft or deformable and/or movable or displaceable examination objects.

The acquisition of the pre-operative 3D data set may refer to a recording of the data set or of raw or measurement data underlying it. Similarly, however, the acquisition of the pre-operative 3D data set may include retrieval or reception of the 3D data set, for example, from a data store that is provided, by a data processing facility by which the method is carried out.

In a further method act of the method, a resection surface, (that is, an incision surface or surface arising and remaining in or on the examination object during the resection), is acquired by ultrasound or ultrasonic scanning to create an ultrasound data set describing or representing the resection surface. Advantageously, an ultrasonic device or an ultrasonic probe or an ultrasonic head used for this is not necessarily arranged directly on the resection surface itself. The scanning or acquisition of the resection surface may thus take place in this context contactlessly in relation to the resection surface, wherein however the ultrasonic probe or the ultrasonic head may lie in another region of the examination object spaced apart thereon from the resection surface, in order to achieve a particularly good imaging quality. Particularly advantageously, the resection, that is the corresponding intervention or the medical personnel is not significantly disturbed or impaired by the ultrasonic device or by the acquisition of the resection surface by ultrasound, so that the intervention does not have to be interrupted for the performance or use of the method proposed here.

In a further method act of the method, the ultrasound data set (and therewith the current or thereby acquired or described resection surface) is registered with the current 3D data set or the corresponding 3D model of the examination object. For this purpose, in principle, registration methods known from the field of medical imaging and image processing may be used.

In a further method act of the method, the respective current 3D data set is updated with the resection surface acquired by ultrasound, that is, with the ultrasound data set. In other words, the acquired resection surface or the course thereof may be entered or inserted into the 3D data set or represented therein. For example, the resection surface may be colored differently from other regions or features or may be represented, for instance, shaded or partially transparent in the 3D data set. Similarly, the 3D data set or the corresponding model of the examination object may be formed according to the resection surface, in other words therefore, the actual resection or the result thereof may be reproduced or represented virtually.

Initially or on a first run-through of the method, the current 3D data set may be the original pre-operative data set. The method may however be carried out once again during the resection or intervention or if new ultrasound data, that is, an updated resection surface is available. Then, the respective last updated 3D data set may be used as the respective current 3D data set. Thus, for the updating of the 3D data set, it is not always necessary to proceed from the original pre-operative 3D data set, but rather an incremental or iterative updating may take place.

In each case when a new or updated ultrasound data set has been generated, (e.g., is available), it may similarly be registered, in each corresponding iteration act or pass of the method, with the respective current 3D data set. Similarly, however, a respective registration with the original pre-operative 3D data set is possible, in particular, because for each iteration or version of the 3D data set, the same coordinate system may be used unchanged.

For the acquisition of the resection surface, a 2D or a 3D ultrasonic device may be used. In particular, where a 2D ultrasonic device is used, it may be acquired or tracked by a tracking system. The tracking system used for this may be used as a navigation system for the respective intervention or may be part of a navigation system of this type. Data acquired or determined by the tracking system regarding the position and, e.g., the orientation, that is, the overall posture of the ultrasonic device used, may be used as a reference for the registration of the ultrasound data set with the 3D data set.

The ultrasound data set may describe or represent the resection surface three-dimensionally. For this purpose, when a 2D ultrasonic device is used, it may be, for example, moved or displaced and thus the resection surface may be acquired from at least two different angles or viewing directions. In conjunction with the position data and possibly orientation data, (that is, posture data, acquired or determined by the tracking system, for the ultrasonic device and/or based on structures visible in the ultrasound data set which may also be visible in the 3D data set), the resection surface may be reconstructed three-dimensionally or the registration of the ultrasound data set may be carried out or supported with the 3D data set.

The method has the advantage that no typically imprecise model assumptions are made for a deformation model and no correspondingly complex calculations or simulations are carried out, because the actual real resection surface may be acquired or reconstructed directly based on the ultrasonic scan or the ultrasound data, that is, ultimately directly based on structures contrasted in the ultrasound data set, and may be registered and combined with the 3D data set.

The use of ultrasound for the acquisition of the resection surface may be gentler as compared with other imaging methods for the respective patient, because, for example, no contrast medium is administered, and no radiation burden occurs. It is also particularly advantageous that the acquisition of the resection surface by ultrasound intraoperatively is possible with less effort and less distraction of or interference with the medical personnel. Through the method, therefore, it may be determined during the resection where the respective current actual incision surface or resection surface extends in the preoperative 3D data set or 3D model. This may be a significant aid to the medical personnel, for example, in order to follow a preoperative planning precisely or to maintain a predetermined spacing from non-visible regions of a tissue region to be resected, for example, a tumor. With this, the method may contribute toward improved oncological outcomes.

With the particularly easy, and thus actually, in a plurality of cases, practicably usable acquisition of the resection surface and the corresponding updating of the 3D data set during the intervention, an improved applicability of navigation systems during interventions on soft deformable and/or movable organs may be achieved.

Although the use of deformation models by the application of the method may be omitted, a modelling or simulation of deformations of the examination object based on or taking account of the ultrasound data set and thus of the real intraoperative course of the resection surface may advantageously be carried out more precisely and reliably than, for example, based on model assumptions which may be used.

In certain examples, the resection surface may be acquired continuously by ultrasound during the resection, that is, without interruption, and the ultrasound data set may be continuously updated accordingly. Such a continuous acquisition of the resection surface does not necessarily refer to a continuous operation of the respective ultrasonic device, but may equally refer to an acquisition of the resection surface with an operating frequency of the ultrasonic device, for example, according to the ultrasound frequency or a current or line frequency, or in a pulsed operation. The resection surface may be acquired with an image frequency or refresh rate of at least 10 Hz or at least 24 Hz. Thereby, for practical purposes, a live or real-time acquisition of the resection surface and accordingly a live or real-time updating of the 3D data set may take place. This may advantageously offer a particularly good support to the medical personnel.

In principle, however, the method may also be used successfully at other ultrasonic image or acquisition frequencies. For example, the resection surface may also be acquired only a few times or once per second, once every 10 seconds, once every 30 seconds, or once per minute or respectively on a corresponding operating action by the medical personnel. The described advantages of the present disclosure may also arise in this or in similar application scenarios.

In a further advantageous embodiment, immediately before the start of the resection, the then still intact examination object is acquired by an imaging modality and corresponding acquisition data which describes or specifies a then current posture of the examination object, is registered in the context of an initial registration with the preoperative 3D data set. In other words, therefore, this initial registration is carried out after the creation of the preoperative 3D data set, but before the start of the resection, that is, before a resection or incision surface forms. The examination object may be acquired, (e.g., imaged), for the initial registration while it is already in its posture intended or planned for the execution of the resection.

Between the creation of the preoperative 3D data set and the resection, the posture of the examination object may change. By the initial registration, such changes or a deformation of the examination object because the creation of the preoperative 3D data set may be acquired particularly reliably and the initial registration and later also the registration of the ultrasound data set with the 3D data set may be carried out particularly reliably and robustly, because possible deviations or differences from the preoperative 3D data set are here reliably attributable to positional changes, movements or deformations of the examination object and not to the resection. By the initial registration, therefore, a particularly robust starting point is provided for the further acts of the method. This may ultimately lead to a greater success of the method, (e.g., an improved accuracy and reliability of the registration of the resection surface with the 3D data set), and thus ultimately may contribute to an improved treatment success. The imaging modality used here may include ultrasound, X-ray imaging, computed tomography, fluoroscopy, magnetic resonance tomography (MRT), and/or the like.

In a further advantageous embodiment, the resection surface or a change thereof is determined by automatic detection of an air gap on the resection surface. In other words, during the resection, a new surface forms, specifically the resection surface on the examination object, wherein this new surface adjoins a surrounding air volume. Such a surface, (that is, a transition from the material of the examination object to air and thus to a corresponding air gap in or on the examination object), behaves differently under ultrasound or influences a propagation and reflection of ultrasonic waves differently from the same material region of the examination object before the resection, that is, while this material region is still situated within the examination object and does not form a boundary surface. Based on these different properties or of the corresponding regional changes of the properties of the examination object, the resection surface, or the change thereof may be detected. For this purpose, for example, an object recognition or feature recognition algorithm, (that is, a corresponding data or image processing program for the automatic recognition or determination of the resection surface or the change thereof), may be used. Particularly advantageously, no direct optical line of sight to the resection surface need exist or be kept clear, because the resection surface may be recognized, for example, based on ultrasonic signals or reflections not from the air side but from a material side or interior side of the resection surface. Thereby, the ultrasonic device or its ultrasonic probe or head may be arranged on a side of the examination object opposite to the resection surface. The resection surface itself need not necessarily be cleaned continuously or regularly, for instance, kept completely free from blood in order to enable the acquisition or determination of the resection surface.

In a further advantageous embodiment, the resection surface is acquired by Doppler ultrasonography, also called Duplex sonography. Based on corresponding Doppler ultrasound data, intact and separated parts of the examination object, (e.g., cut off from a blood supply), are differentiated from one another. This differentiation is then used as a boundary condition in the registration of the ultrasound data set or of the resection surface with the 3D data set. In other words, therefore, different Doppler shifts are evaluated, (e.g., automatically), in order to identify the resection surface particularly accurately and reliably. This may improve, or make more robust, the correct determination of the resection surface, particularly if a separated part of the examination object, that is, a respective resected part is still within a region acquired or imaged by the ultrasound. In such a case, a plurality of newly arising surfaces may be acquired and detected by the ultrasound, wherein the different Doppler properties of these surfaces or material or tissue regions bordering thereon, in particular, respective blood vessels extending therein or bordering thereon, but advantageously enable a correct assignment of a known or detected surface to the resection surface, or as the resection surface. A blood flow may be detected, in particular, by Doppler ultrasound. On separation or parting of a vessel or a piece of a vessel by the resection, the blood flow in this vessel or piece of a vessel will change, in particular dry up, as compared with intact tissue or an intact vessel. This is then detectable based on the recorded Doppler ultrasound data, so that based thereon, a differentiation of intact tissue and a resected part may be carried out. In this way, advantageously without additional devices and without additional effort on the part of the medical personnel, false detection or false registrations may be particularly reliably prevented or at least reduced. Thus an enhanced safety and thus also an improved support for the medical personnel may be achieved during the registration.

In a further advantageous embodiment, the registration of the ultrasound data set with the 3D data set is carried out based on at least one spacing of structures visible in the ultrasound data set or at least one visible, that is, imaged feature. Thereby, the spacing of the ultrasonic probe or the ultrasonic head used from one or more structures or features and/or a spacing of a plurality of structures and/or features from one another, as it appears in the ultrasound data set may be used. Structures or features positioned locally, that is, closer to the resection surface or closer to a region of a respectively current change of the resection surface, or their spacings, may be prioritized as compared with further removed structures or features, or the spacings thereof. For example, exclusively structures or features which, where present, are positioned within a predetermined distance from the ultrasonic device or from one another or from the resection surface may be used, or a registration or overlay accuracy for such structures and/or features may be more strongly weighted than for further removed structures or features. If the ultrasound data set and the 3D data set therefore cannot be brought into coincidence, for example, in all regions or points, the greatest possible congruence or overlaying of local structures as compared with more remotely positioned structures is prioritized. This is particularly advantageous because the resection itself is a local procedure and an exact position of the resection surface may be of greater significance for treatment success than, for example, a deformation in a subregion of the examination object positioned remotely from the resection surface.

Overall, the registration may thus be particularly relevant and may take place in the region of the greatest interest particularly robustly and reliably, in particular without a complex deformation model having to be used.

The structures or features used here may be anatomical and/or synthetic. Thus, for example, vessels, vessel branches, edges of tissue regions, in particular, of a tumor or an organ, but also markers or markings applied to the examination object for the respective intervention may be used. Structures or features are used here, which are visible not only in the ultrasound data set but also in the 3D data set and/or under an imaging modality that is registered to the 3D data set, may be used during the intervention for imaging the examination object, for example, an intraoperative CT.

The spacings observed here are used, as they appear in the ultrasound data set and may thus (at least apparently) be dependent upon a viewing or observation angle. Therefore, the spacings offer a possibility for carrying out the registration correctly not only with regard to the position of a structure or a feature, but also with regard to an orientation or alignment. In particular, when body structures or features of the examination object are used, advantageously, additionally an inaccuracy or error source in the positioning of corresponding artificial structures or features may also be prevented.

In a further advantageous embodiment, the registration of the ultrasound data set with the 3D data set is carried out anew when an updated ultrasound data set has been created or, for example, when a change to the resection surface or the ultrasound data has been detected. The renewed registration is carried out, in each case, based on the respective preceding registration, that is, the registration that was valid until the respective renewed registration. In other words, the renewed registration therefore need not be carried out from scratch, that is, without prior knowledge, but rather previous registration data may be used as a reference or starting point. For example, changes may be determined in comparison with the preceding registration or the preceding or previous ultrasound data set and thus the registration (and finally also the 3D data set) may be incrementally modified or updated. This may advantageously lead to a particularly reliable and robust registration because, for example, on each renewed registration, only relatively small changes relative to the respective preceding registration are compensated for or taken into account as compared with a registration to the originally unchanged preoperative 3D data set and smaller uncertainties and fewer error sources may be associated with such smaller changes or modifications.

The respective preceding registration may thus be used, for example, as a boundary condition for a coarse alignment of the ultrasound data set relative to the 3D data set, if relevant, dependent upon a time interval which has elapsed because the respective last registration. It may thus be implausible that during a particular time interval, a particular especially large change, for example, lying above a predetermined, where relevant, time-dependent threshold value has taken place. If an excessively large change of this type is detected, this may be interpreted as a faulty detection or identification of a feature or used as an indication thereof. In this way, the accuracy of the intraoperative registration of the ultrasound data set to the 3D data set as a whole may be improved.

In a further advantageous embodiment, for the registration of the ultrasound data set with the 3D data set and/or for the updating of the 3D data set, a planning data set that is provided, (e.g., predefined), is used as a boundary condition for plausibility checking the registration or the updating. The planning data set therein describes a preoperative planning for a spatial course of the resection. For example, a threshold value for a maximum spatial deviation of the acquired or detected resection surface from its planned course according to the planning data set may be defined. If this threshold value is at least apparently exceeded, this may possibly suggest a faulty detection or faulty identification of features or structures used for the registration or updating. In such a case, the respective registration or updating may be checked or rejected in order advantageously to improve altogether the accuracy and reliability of the registration or the updating.

Similarly, the planning data set may be used as a restriction or as a template for an initial rough allocation or rough set-up of the ultrasound data set relative to the 3D data set, because it may be assumed that the actual resection will not take place in a completely different region of the examination object than planned. In this way, for example, a faulty automatic registration or updating which may arise in that different subregions of the examination object have features or structures that are the same as, or very similar to, one another or, for example, an ambiguity arises with regard to a rotation of the ultrasound data set relative to the 3D data set, may be prevented.

For example, before the updating of the 3D data set, the acquired resection surface, that is, the ultrasound data set and/or a detected change in the resection surface may be checked or plausibility checked based on the planning data set. It may then be provided that the updating of the 3D data set according to the current ultrasound data set is only actually carried out when the automatically determined resection surface or the course or changing thereof is plausible, that is, for example, deviates by not more than a defined value from the planning data set. For example, excessively large jumps or positional changes of the resection surface or the like, that are, for instance, above the defined threshold value, may be automatically classified as implausible and thus rejected.

In a further advantageous embodiment, a position or a posture of an instrument provided for performing the resection and/or an operating state of the instrument is automatically acquired and tracked. An enlargement of the resection surface is only entered or represented in the 3D data set for the updating thereof if at the point in time of the detected enlargement of the resection surface, the instrument, (e.g., in the region of the hitherto acquired resection surface), was in contact with the examination object or in contact with the previous resection surface or with a thereto adjoining region of the examination object and/or if the operating state of the instrument at this point in time is or was consistent with the enlargement of the resection surface.

Whether the position of the instrument in mechanical contact with the examination object or its operating state is being used or prioritized here as a criterion may depend, in particular, on the type of the respective instrument. This may be provided in advance, for example, as a parameter value or an input data item, that is, defined in advance.

The operating state of the instrument may be a switching state or an actuating level of an operating element, for example, a foot pedal, an energy supply to the instrument with operating energy, a measure or a strength of this energy supply such as an actual current flow or an actually drawn electric power, a rotary speed, or a load of a motor or drive or of another component of the instrument, and/or the like or may be described or characterized thereby.

If the instrument is, for example, an energy instrument based, for instance, on ultrasound, plasma or laser or based on the material-influencing effect of other electromagnetic waves, then it may be assumed that an actual resection and thus an enlargement of the resection surface may only occur when the instrument or its energy supply is active or switched on.

Similarly, a speed or a rate of the enlargement of the resection surface, that is, a progress or a progress speed of the resection based on the energy quantity or power fed to the instrument or the values of the other specified parameters are plausibility checked, for example, because thereby a cutting performance or tissue removal performance of the instrument may be limited.

If, however, for example, a mechanical instrument is used, then it may be assumed that an enlargement of the resection surface may actually only occur when the instrument is actually in mechanical contact with the examination object.

Detected enlargements or changes to the resection surface that are not plausible according to these criteria may then automatically be classified as faulty detection and rejected. In this way, the registration and updating of the 3D data set may be carried out particularly reliably and robustly and thus a quality of the support offered to the medical personnel may be improved. Because an output of implausible data or a corresponding visualization may be thus prevented or minimized, a faulty item of information or a confusion of the medical personnel may accordingly be prevented or minimized, which ultimately may contribute to the treatment success.

In a further advantageous embodiment, after the resection, a second ultrasound data set is acquired and recorded by moving an ultrasonic probe or an ultrasonic head along the resection surface in direct mechanical contact with it. In other words, ultrasound data which represents the examination object in a state after the resection and which is or has been obtained in that the actual physical resection surface which has arisen as the result of the resection is scanned or sampled with the ultrasonic probe or the ultrasonic head directly, that is, in direct contact, may thus be acquired here. The second ultrasound data set is then registered with the 3D-data set of the examination object. This may take place as described in relation to the first ultrasound data set, for example, taking account of structures visible in the second ultrasound data set or the like, e.g., with local prioritizing or overweighting. Thereby, the second ultrasound data set may be registered with the original preoperative 3D data set or with the most current, that is, last updated 3D data set or with an overlaying or combination of these.

Based on the second ultrasound data set registered to the 3D data set, and the 3D data set, a course of the resection surface is then checked or evaluated as the result of the resection. This may take place in comparison with or in relation to a preoperative planning, (e.g., a planned result model of the examination object planned with the planning data set or a defined planned result model of the examination object).

The acquisition of the second ultrasound data set may include its reception or reading in by a data processing facility used or provided for the method, in particular, for the checking. The actual physical scanning or sampling of the resection surface with the ultrasonic probe or the ultrasonic head (insofar as it relates to or includes a surgical act) is thus not additionally claimed here.

The checking of the result of the resection directly after the resection and possibly making use of devices already present in the operation region advantageously enables, without significant disruption of the work process, to recognize whether the planned resection was, for example, incompletely performed or was successful, at least according to the planning. Advantageously, a repeat resection, (that is, an improvement), may then be carried out directly and without delay in order to achieve an optimum treatment result. Such a direct checkability of the oncological result after or at the end of the resection is thus of particular interest and advantage for the wellbeing of the patient. In that the ultrasonic probe or its ultrasonic head for recording the second ultrasound data set is arranged directly on the resection surface, a further reference, that is, a further boundary condition or further position data for determining the actual course of the resection surface, is provided so that, in this way, the resection surface may be recognized particularly accurately and reliably.

A position or posture of the ultrasonic probe or the ultrasonic head may thereby be tracked during the guidance along the resection surface by a tracking system, for example, the aforementioned tracking or navigation system. This therefore provides a position data set for the ultrasonic probe which, however, due to the direct mechanical contact of the ultrasonic probe with the resection surface, also describes the course of the resection surface. The resection surface may thus be determined based on this position data set and additionally based on the second ultrasound data set, whereby advantageously, a particularly high degree of reliability and accuracy in the determination of the resection surface and in its registration to the 3D data set may result. Particularly advantageously, the tracking of the ultrasonic probe enables the use of a 2D ultrasonic probe which may advantageously be smaller, less expensive, and higher resolution than a 3D ultrasonic probe. Thus, the resection surface may possibly be scanned or sampled in a particularly detailed manner, particularly exactly and also with restricted space.

In an advantageous development, for the checking, a spacing of the resection surface from a region to be resected according to a preoperative planning, (e.g., according to the specified planning data set), is determined. This region to be resected may be a particular defined tissue region, e.g., a tumor or the like, possibly including a predetermined environment, a region of interest (RoI), or the like. The spacing may then be related, for example, to an outer edge such as closest to the resection surface, or to a center point of the region to be resected. The spacing determined is then evaluated according to a defined criterion. Where present, at least such regions of the resection surface for which the criterion is not met are marked automatically in the second ultrasound data set and/or in the 3D data set or in their superposition or combination. Similarly, regions in which the criterion is met may be marked. Additionally, by the marking, a level of the non-fulfilment or fulfilment of the criterion may be indicated.

The criterion may be whether a defined threshold value for the spacing is achieved, exceeded, or undershot. It may thus be evaluated and marked accordingly whether or where insufficient or excessive material of the examination object has been removed during the resection. The marking may take place with a coloration according to a defined color scale, by displaying numerical values or symbols, and/or the like. A corresponding marking may be displayed by a virtual or augmented reality or a corresponding device, for example, a pair of data glasses or a head-mounted display (HMD), or the like.

Therefore, a deviation of the actual result of the resection from a preoperative planning may thus be visualized, whereby a deviation of this type may be determined particularly reliably and clearly or may be perceived by the medical personnel. Thus, the medical personnel may be supported particularly reliably and effectively in the avoidance or reduction of treatment errors, for example, an incomplete resection.

In an advantageous development, after the resection, a surface of a respective resected part is additionally acquired. For this purpose, for example, the surface of the resected part is also scanned or sampled in contacting manner with the ultrasonic probe or the ultrasonic head and/or the resected part is imaged by an imaging modality. A corresponding resected part data set describing or representing the resected part, (e.g., its surface, shape, size, and/or the like), is then also taken into account or evaluated in the checking or for the evaluation of the result of the resection. In this way, the checking or evaluation may be carried out particularly reliably and robustly because more data is available. Thus, inaccuracies in the acquisition of the resection surface may be reduced based on the corresponding acquired surface of the resected part. The resected part may be acquired, without difficulty, completely from all sides, which may not be the case on the resection surface due to restricted space conditions and the need to avoid an injury to the examination object.

A further aspect of the present disclosure is a system for supporting medical personnel during a resection. The system has a data processing facility which is designed and configured to carry out at least one embodiment of the method. The system may thus be the aforementioned system. The system may have some or all of the parts, components and/or properties and the corresponding advantages described in relation to the method.

A further aspect of the present disclosure is a computer program product which includes commands or control instructions which on their execution by a computer, in particular, by a system or its data processing facility, cause this computer or this system to carry out at least one embodiment of the method, or to bring about its execution. The computer program product may be a computer program which implements, that is, encodes or represents these commands. Similarly, the computer program product may be a computer-readable data carrier on which a computer program of this type, that is corresponding program code, is stored. The method or its method acts may thus be computer-implemented in whole or in part.

For the execution of the method, the system or its data processing facility may include a computer program product. In particular, the data processing facility of the system may have a processor and a data store associated therewith, in particular, a data carrier. By the processor, (e.g., a microprocessor, microchip, microcontroller, a hardware circuit, and/or the like), the computer program which implements the method and is stored on the data store may be executed.

The data processing facility of the system or the system itself may additionally have further components, in particular one or more data interfaces for receiving and/or outputting data of, for example, the ultrasound data set, the 3D data set, the result or the evaluation of the resection, and/or the like. Furthermore, the system may include further components or parts, (e.g., the aforementioned ultrasonic device, the aforementioned instrument, the aforementioned imaging modality, and/or the like).

The properties and developments as set out above and in the following of the method, the computer program product and the system and also the corresponding advantages are each analogously and reciprocally transferable between these aspects of the disclosure. Such developments of the aspects which have embodiments which, for the avoidance of unnecessary redundancy, are not explicitly described here in the respective combination or are not separately described for each aspect of the disclosure, thus also belong to the disclosure.

### BRIEF DESCRIPTION OF THE DRAWINGS

Further features, details, and advantages of the present disclosure are provided in the following description of exemplary embodiments and are illustrated in the drawings, in which:

FIG. 1 is an exemplary schematic flow diagram of a method for supporting medical personnel during a resection.

FIG. 2 is an example of a schematic overview to illustrate the method and facilities for its execution.

FIG. 3 is an exemplary schematic representation to illustrate a 3D data set of an examination object.

FIG. 4 is an example of a schematic overview to illustrate a further part of the method.

The components of the embodiment variants as described in the exemplary embodiments each represent individual features of the disclosure that are to be regarded as independent of one another and each also further develop the disclosure independently of one another and are thus also to be considered individually, or in a different combination from that shown, as a constituent of the disclosure. Furthermore, the embodiment variants described are also enhanceable with others of the previously described features of the disclosure.

In the drawings, details having the same function or corresponding to one another are each provided with the same reference signs.

### DETAILED DESCRIPTION

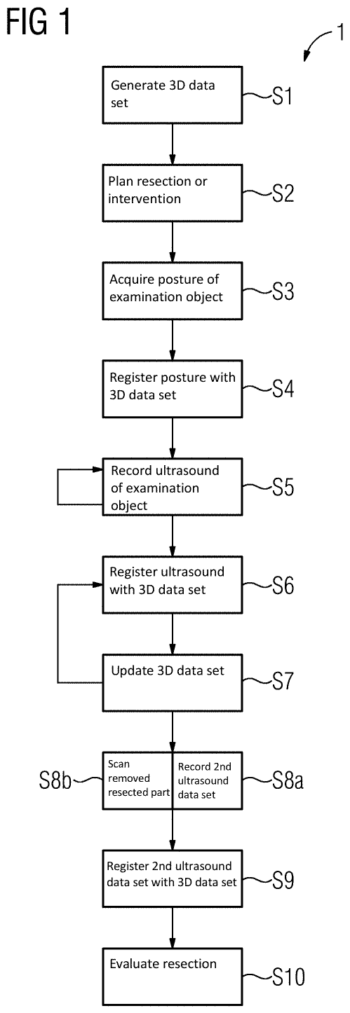

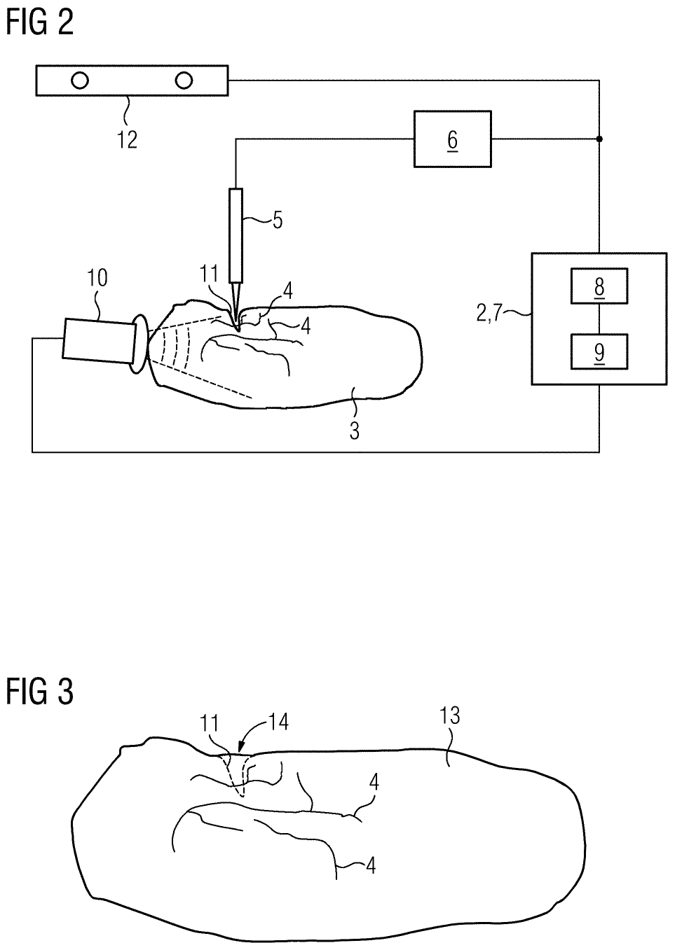

FIG. 1 shows by way of example, a schematic flow diagram for a method for supporting medical personnel during a resection. Method acts S1 to S10 carried out thereby will now be described in greater detail referring to the other drawings. FIG. 2 shows a schematic overview to illustrate a situation during the execution of the method and of devices and facilities used therein.

In particular, for carrying out the method, a system 2 is used which includes at least one data processing facility 7. This data processing facility 7 itself herein includes a processor 8 and a data store 9 associated therewith. The method may be implemented as a computer program which may then be stored on the data store 9. This computer program, that is, corresponding program code may then be executed for carrying out the method by the processor 8. At least some of the method acts S1 to S10 represented in FIG. 1 may thus represent corresponding commands, control instructions, program modules or the like of the corresponding computer program.

The data processing facility 7 may be a separate facility, (e.g., a computer). Such a computer may be arranged, for example, locally at an execution location of the method or the resection possibly carried out in parallel. Similarly, the computer or data processing facility 7 may be situated on site or may be part of a, possibly cloud-based, computing center. Similarly, the data processing facility 7 may integrated into an imaging device, (e.g., an X-ray device, an MRT system, or the like). Such a device or system may then represent the system 2 or a part of the system 2.

In the present example, the resection is to be carried out on an examination object 3 which may be a liver of a patient (not shown here). The examination object 3 has structures 4 that may be both anatomical features and also synthetic markers or markings or the like. For the performance of the actual resection, a resection instrument 5 indicated here schematically may be used. The resection instrument 5 may be designed in a variety of ways and make use of different functional principles, depending upon need and the application case. Thus, the resection instrument may be configured for removing or separating biological tissue and thereby, for example, in an energy-based or purely mechanical manner. For the supply of the resection instrument with operating energy, it is connected to an energy supply unit 6. The energy supply unit 6 may have electrical components and/or an operating element for activating and deactivating the resection instrument 5. The energy supply unit 6 may be or include a foot pedal.

Furthermore, an ultrasonic device is provided, which is represented here by an ultrasonic probe 10 connected to the data processing facility 7. At the point in time shown in FIG. 2, the ultrasonic probe 10 is arranged laterally on the examination object 3 in order at least partially to acquire, (e.g., image), the examination object 3. The ultrasonic probe 10 is thereby arranged or, in particular, oriented such that it acquires or images a region of action of the resection instrument 5 and acquires or images such structures 4 as are situated in the environment thereof. In particular, by the ultrasonic probe 10, a resection surface 11 arising during use of the resection instrument 5, (e.g., during the resection), on the examination object 3 is acquired, (e.g., imaged).

Furthermore, a tracking system 12 is also provided here. The tracking system 12 herein includes a camera for optically acquiring a position and, e.g., a posture of the resection instrument 5 and of the ultrasonic probe 10. By this camera and/or of further (e.g., electromagnetic) tracking facilities of the tracking system 12, the resection instrument 5 and the ultrasonic probe 10 may be tracked, (e.g., monitored), with regard to their position or posture.

The instrument 5, the energy supply unit 6, the ultrasonic probe 10, and/or the tracking system 12 may each be separate facilities connected to the system 2 or the data processing facility 7 or may be part of the system 2. In particular, in the latter case, at least some functions of these facilities may be assumed or performed by the data processing facility 7. This may include a processing of ultrasound data provided by the ultrasonic probe 10 and/or position data or sensor data, (e.g., camera images), provided by the tracking system 12. Similarly, an operating state of the resection instrument 5 or of the energy supply unit 6 may be acquired or monitored by the data processing facility 7.

In the method act S1, a preoperative 3D data set, (e.g., a virtual model of the examination object 3), is generated, (e.g., by the data processing facility 7). For this purpose, the examination object 3 may be imaged initially by an imaging modality and raw data recorded thereby may be processed by the data processing facility 7 to the preoperative 3D data set. Similarly, the preoperative 3D data set may be already prepared and may be received or retrieved by the data processing facility 7.

In the method act S2, the resection or a corresponding intervention is planned, or a corresponding planning data set is acquired by the data processing facility 7. Such a planning data set may specify or describe, for example, a respective region of interest (RoI), a region to be resected, a planned final course of the resection surface 11, a success criterion for evaluating the resection or a result of the resection or intervention and/or the like. For the success criterion, for example, a spacing to be maintained from the region of interest or its center and/or from at least one anatomical or synthetic feature, for example, one of the structures 4, that is, for example from a vessel, a tissue or an organ boundary or the like may be defined.

In the method act S3, immediately before the start of the resection, the examination object 3 on which the resection is to be performed is acquired in its then current posture, for example, by the ultrasonic probe 10 and/or an imaging modality, (e.g., an X-ray device (not shown here in detail)), which may be part of the system 2.

In the method act S4, an initial rigid or deformable registration of the current posture of the examination object 3 acquired in the method act S3 to the preoperative 3D data set is carried out by the data processing facility 7.

In the method act S5, an ultrasound recording of the examination object 3, (in particular, the resection surface 11 and a region surrounding it), is continuously or regularly made by the ultrasonic probe 10. In other words, a progress of the resection or a creation and enlargement of the resection surface 11 is therefore continuously observed with surgical ultrasound.

In the method act S6, ultrasound data recorded in method act S5 by the ultrasonic probe 10 or an ultrasound data set generated therefrom is registered with the 3D data set. This may accordingly be carried out automatically by the data processing facility 7 continuously or regularly or respectively on detection of changes of the resection surface 11, for example, going beyond a defined threshold value.

Thereby, initially, a plausibility checking on the basis, for example, of the planning data set of a respective preceding registration and/or an earlier version of the 3D data set may be carried out automatically. The registration may only be carried out if the plausibility checking, for example, of the resection surface 11 detected in or based on the ultrasound data or of its detected change was successful.

The resection surface 11 may be detected or determined in the ultrasound data set, on the basis, in particular, of an air gap arising externally at the resection surface 11.

The registration may be carried out, in particular, by a determination of a local spacing from the structures 4 and/or based on a respective current or preceding registration.

Based on the ultrasound data acquired with the ultrasonic probe 10, a repeated registration of a current state of the examination object to the 3D data set therefore takes place here. For the support of the registration and possibly the plausibility checking, the resection instrument 5, and the ultrasonic probe 10 are likewise continuously or regularly tracked by the tracking system 12.

On successful plausibility checking, in method act S7, based on the respective current ultrasound data set, an updating of the 3D data set takes place with the respective current or new state of the examination object 3, in particular, of a respective current or new course of the resection surface 11. If corresponding data is not plausible and the plausibility checking has failed, for example, the ultrasound data acquired since the last ultrasound recording or the last successful registration or updating may be rejected.

FIG. 3 shows a schematic representation for illustration of the 3D data set of the examination object 3. For this, a 3D model 13 of the examination object 3 is shown. In the present case, both an original surface 14 of the examination object 3 as it was before the start of the resection, and a current course of the resection surface 11 are both shown. Based on the 3D model 13, the current course of the resection surface 11, (e.g., relative to the structures 4), are recognizable particularly easily and clearly for respective medical personnel. By the 3D model 13 updated a plurality of times or continuously, that is, the repeated or continuous correction of the acquired resection surface 11 or its course and the corresponding updating of the 3D model 13, an effective support may thus be provided to the medical personnel based on the ultrasound data.

As is indicated here by respective loop-structured program or sequence paths, the method acts S5 to S7 may be repeated or run through a plurality of times or continuously.

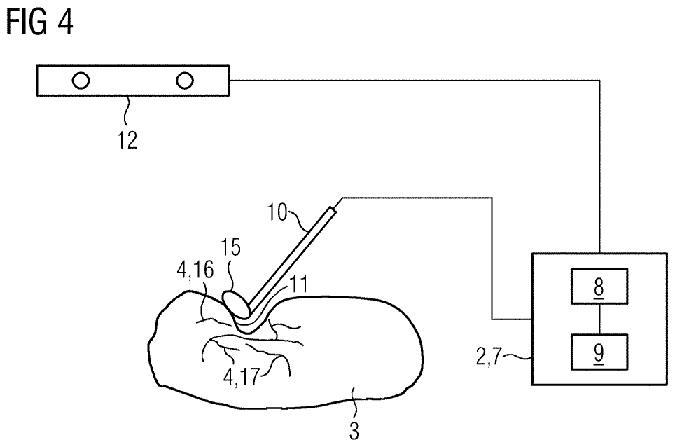

After the resection, a checking and evaluation of its result is provided. For this purpose, FIG. 4 shows a further schematic overview with the system 2 and the examination object 3, here however in a state after the resection, as is recognizable based on the enlarged resection surface 11.

In the method act S8a, after the resection, a second ultrasound data set of the examination object 3 is recorded by the ultrasonic probe 10. For this purpose, an ultrasonic head 15 of the ultrasonic probe 10 is guided along the resection surface 11 in direct mechanical contact and thus scans the resection surface 11 by the ultrasonic head 15. Thereby, the posture of the ultrasonic probe 10 or of the ultrasonic head 15 is tracked by the tracking system 12. Optionally, in the method act S8b, a resected part removed from the examination object 3 (not shown here) is likewise scanned by the ultrasonic probe 10 and the ultrasonic head 15 in a corresponding manner and is thus acquired by ultrasound.

In the method act S9, the second ultrasound data set, possibly including an ultrasound resected part data set describing or representing the resected part is registered with the most current 3D data set and/or with the preoperative 3D data set. This may be carried out, for example, automatically by the data processing facility 7.

As previously for the registration or registrations in the method act S6, a local, that is, position-dependent weighting of the registration may be used. Different structures 4 may thereby be handled differently. Thus, for example, a registration accuracy for local structures 16 that are situated more closely to the resection surface 11 may be prioritized, as compared with a registration accuracy for remote structures 17 that are situated at a greater distance from the resection surface 11. This may be advantageous in particular in the case of deformable organs, (e.g., the liver), and may enable a complex global deformation model for the respective overall organ to be dispensed with.

In the method act S10, the result of the resection may be checked or evaluated based on the second ultrasound data set, possibly the resected part data set and the 3D data set as well as possibly the planning data set. This may be carried out automatically by the data processing facility 7. Herein, the success criterion specified or defined in the method act S2 may be checked. Thus, for example, a spacing of the resection surface 11 from the structures 4, from the original surface 14 or from a center of a recess or the like arising due to the resection in or on the examination object 3 may be compared with a defined spacing threshold value. A respective result of the evaluation or checking may also be visualized automatically by the data processing facility 7, for example, in the 3D model 13 and/or by virtual or augmented reality. A recommendation for corrections to the resection may be output by the data processing facility 7 for the regions in which the resection surface 11 does not meet the defined success criterion.

Overall, the examples described herein show how, by a live generation of the resection surface 11 by ultrasound and by a subsequent ultrasound sweep over the resection surface 11 for success or spacing evaluation, with regard, for example, to tumor borders, medical personnel may be supported technically during an intervention.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for supporting medical personnel during a resection, the method comprising:
acquiring a preoperative three-dimensional (3D) data set of an examination object on which the resection is to be carried out;
acquiring a resection surface by ultrasound to generate an ultrasound data set describing the resection surface;
registering the ultrasound data set with the 3D data set; and
updating the 3D data set with the resection surface.

2. The method of claim 1, wherein the resection surface is acquired continuously by ultrasound during the resection and the ultrasound data set is continuously updated accordingly.

3. The method of claim 1, further comprising:
acquiring, immediately before the start of the resection, acquisition data of the examination object by an imaging modality, wherein the acquisition data describes a then current posture of the examination object; and
registering, in a context of an initial registration, the acquisition data with the preoperative 3D data set.

4. The method of claim 1, wherein the resection surface or a change of the resection surface is determined by automatic detection of an air gap on the resection surface.

5. The method of claim 1, wherein the resection surface is acquired by Doppler ultrasound based on corresponding Doppler ultrasound data,
wherein intact and separated parts of the examination object are differentiated from one another, and
wherein the differentiation is used as a boundary condition in the registration of the ultrasound data set with the 3D data set.

6. The method of claim 1, wherein the registration of the ultrasound data set with the 3D data set is carried out based on at least one spacing of structures visible in the ultrasound data set, and
wherein structures positioned closer to the resection surface or to a region of a respective current change to the resection surface are preferred as compared with structures positioned further removed.

7. The method of claim 1, wherein the registration of the ultrasound data set with the 3D data set is repeated when an updated ultrasound data set has been generated, and
wherein the repeated registration is carried out based on a respective preceding registration.

8. The method of claim 1, further comprising:
providing a planning data set describing a preoperative planning for a spatial course of the resection; and
using the planning data set as a boundary condition for plausibility checking of the registration of the ultrasound data set with the 3D data set and/or the updating of the 3D data set.

9. The method of claim 1, wherein a position of an instrument provided for carrying out the resection and/or an operating state of the instrument is automatically tracked, and
wherein an enlargement of the resection surface in the 3D data set for updating thereof is only entered when, at a point in time of a detected enlargement, the instrument was in contact with the examination object and/or the operating state of the instrument was consistent with the enlargement of the resection surface.

10. The method of claim 1, further comprising:
acquiring, after the resection, a second ultrasound data set by moving an ultrasonic probe along the resection surface in direct mechanical contact with the ultrasonic probe;
registering the second ultrasound data set is registered with the 3D data set of the examination object; and
checking a course of the resection surface based one the registered second ultrasound data set and the 3D data set.

11. The method of claim 10, wherein one position or posture of the ultrasonic probe is tracked by a tracking system while the ultrasonic probe is guided along the resection surface.

12. The method of claim 11, wherein, for the checking, a spacing of the resection surface from a region to be resected according to a preoperative planning is determined and evaluated according to a defined criterion and, where present, regions of the resection surface for which the criterion is not met are automatically marked in the second ultrasound data set and/or in the 3D data set.

13. The method of claim 12, wherein, after the resection, a surface of a respective resected part is additionally acquired and a corresponding resected part data set describing the resected part is taken into account during the checking.

14. The method of claim 10, wherein, for the checking, a spacing of the resection surface from a region to be resected according to a preoperative planning is determined and evaluated according to a defined criterion and, where present, regions of the resection surface for which the criterion is not met are automatically marked in the second ultrasound data set and/or in the 3D data set.

15. The method of claim 10, wherein, after the resection, a surface of a respective resected part is additionally acquired and a corresponding resected part data set describing the resected part is taken into account during the checking.

16. A system for supporting medical personnel during a resection, the system comprising:
a data processing facility configured to:
acquire a preoperative three-dimensional (3D) data set of an examination object on which the resection is to be carried out;
acquire a resection surface by ultrasound to generate an ultrasound data set describing the resection surface;
register the ultrasound data set with the 3D data set; and
update the 3D data set with the resection surface.

17. A computer program product comprising commands which, during execution of the commands by a computer of a system, cause the system to:
acquire a preoperative three-dimensional (3D) data set of an examination object on which a resection is to be carried out;
acquire a resection surface by ultrasound to generate an ultrasound data set describing the resection surface;
register the ultrasound data set with the 3D data set; and
update the 3D data set with the resection surface.
